(19) United States
(12) Patent Application Publication
(10) Pub. No.: US 2017/0319876 A1
(43) Pub. Date: Nov. 9, 2017

(54) CONDITIONING HAIR CLEANSING AGENT

(71) Applicant: Henkel AG & Co. KGaA, Duesseldorf (DE)

(72) Inventors: Dirk Hentrich, Hamburg (DE); Corinna Bender, Hamburg (DE)

(73) Assignee: Henkel AG & Co, KGaA, Duesseldorf (DE)

(21) Appl. No.: 15/444,056

(22) Filed: Feb. 27, 2017

Related U.S. Application Data
(63) Continuation of application No. PCT/EP2015/069395, filed on Aug. 25, 2015.

(30) Foreign Application Priority Data
Aug. 29, 2014 (DE) 10 2014 217 311.6

(51) Int. Cl. A61Q 5/02; A61K 8/46; A61K 8/36; A61K 8/42; A61Q 5/12; A61K 8/73

(52) U.S. Cl. CPC A61Q 5/02; A61Q 5/12; A61K 8/731; A61K 8/36; A61K 8/42; A61K 8/463

(57) ABSTRACT

A hair-cleaning agent includes (in relation to the total weight thereof): a) between 6 and 12 wt. % at least one anionic surfactant; b) between 0.1 and 5 wt. % at least one non-ionic surfactant; c) between 0.01 and 2 wt. % at least one cationic conditioning polymer; and d) (i) between 0.01 and 3 wt. % calcium lactate or (ii) between 0.1 and 5 wt. % lactic arid and between 0.05 and 3 wt. % at least one inorganic calcium salt; and a pH value in the region between 4.3 and 5.0. The hair-cleaning agent is suitable for improving de-tangling properties and combability, as well as reducing electrostatic charging (flyaway hairs) and the occurrence of unruly hair or unwanted crimped hairs (anti-frizz effect), in particular for damaged hair.

### FIELD OF THE INVENTION

The present invention generally relates to cosmetics, and further relates to the use of the cleansing agents for improving detanglability and combability, in particular of damaged hair, and for reducing the static charge of hair and the development of shaggy or undesirably frizzy hair.

### BACKGROUND OF THE INVENTION

The structure of hair can be damaged as a result of environmental factors (such as intensive solar radiation), mechanical stresses (such as intensive combing while applying heat from a hair dryer), or physical or chemical treatments (such as dying, reshaping or straightening the hair). The consequences are often times split ends, hair breakage and/or lack of luster. Damaged hair moreover is difficult to detangle and style.

Hair cleansing agents generally comprise (anionic) surfactants. These are desirable and necessary to be able to remove sebum and other dirt deposits from the surface of the hair and the scalp. However, they also cause lipids and proteins to be removed from the hair or the scalp during cleansing, so that a subsequent treatment with nourishing agents for regenerating the hair is necessary.

Cleansing hair that has already become damaged is therefore problematic, and a need exists for particularly mild hair cleansing agents that do not place an additional burden on the damaged hair during cleansing and nourish the hair.

The formulation of so-called conditioning hair cleansing agents is known. EP 1329215, for example, proposes nourishing hair cleansing compositions that, when diluted 20-fold, have a pH value in the range of 1 to 4 and comprise an anionic sulfate surfactant, a cationic guar gum, a hydroxycarboxylic acid (glycolic acid, lactic acid or malic acid) and a salt (in particular inorganic or organic sodium salts). The hair cleansing agents exhibit particularly good foaming properties and impart more softness, luster and suppleness to hair cleansed therewith.

This technique, however, is not useful, especially when treating damaged hair and/or a dry scalp, since the high acidity of the agents can additionally cause the scalp to dry out and result in undesirable dandruff formation. Moreover, the ability to detangle in particular long, damaged hair was not satisfactory after the treatment with the agents.

A continued need therefore exists for mild hair cleansing agents that are gentle on the scalp and that in particular are suitable for cleansing damaged hair and imparting advantageous visual and haptic properties.

It is therefore desirable to provide mild, nourishing hair cleansing agents that are tolerated well by the skin and hair and mitigate hair damage. In particular long, damaged hair is to be strengthened in terms of the structure thereof during cleansing and should be easily detangled after cleansing.

Furthermore, other desirable features and characteristics of the present invention will become apparent from the subsequent detailed description of the invention and the appended claims, taken in conjunction with this background of the invention.

### BRIEF SUMMARY OF THE INVENTION

A hair cleansing agent includes, based on the weight thereof: 6 to 12 wt. % of at least one anionic surfactant; 0.1 to 5 wt. % of at least one non-ionic surfactant; 0.01 to 2 wt. % of at least one cationic nourishing polymer; and (i) 0.01 to 3 wt. % calcium lactate or (ii) 0.1 to 5 wt. % lactic acid and 0.05 to 3 wt. % of at least one inorganic calcium salt, characterized in that the hair cleansing agent has a pH value in the range of 4.3 to 5.0.

### BRIEF DESCRIPTION OF THE DRAWINGS

The present invention will hereinafter be described in conjunction with the following drawing figures, wherein like numerals denote like elements, and

FIG. 1 is a graph depicting comparative data for wet and dry hair properties following treatment with compositions according to the present invention and also comparative compositions.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

It was found that the skin tolerability and mild nature of nourishing hair cleansing agents can be significantly increased if these comprise a mixture of mild anionic and non-ionic surfactants, cationic polymers, and a special salt (or a special acid/salt mixture) and have a slightly acidic pH value.

A first subject matter of the invention is a hair cleansing agent that, based on the total weight thereof, comprises
a) 6 to 12 wt. % of at least one anionic surfactant;
b) 0.1 to 5 wt. % of at least one non-ionic surfactant;
c) 0.01 to 2 wt. % of at least one cationic nourishing polymer; and
d) (i) 0.01 to 3 wt. % calcium lactate or
   (ii) 0.1 to 5 wt. % lactic acid and 0.05 to 3 wt. % of at least one inorganic calcium salt,
      and has a pH value in the range of 4.3 to 5.0.

The hair cleansing agents according to the invention have a number of advantages: they are especially mild and do not burden or damage the (dry) scalp. Moreover, they impart improved detanglability and combability to the hair treated therewith, both when wet and when dry, and reduce the static charge and “fly-away” of hair as well as the development of shaggy or undesirably frizzy hair (“anti-frizz effect”).

The hair cleansing agents according to the invention preferably comprise the active ingredient combination a) to d) in a cosmetically acceptable carrier. A cosmetically acceptable carrier within the scope of the invention shall preferably be understood to mean an aqueous or aqueous-alcoholic carrier. Particularly preferably, the cosmetic carrier includes at least 50 wt. %, more preferably at least 60 wt. %, and particularly preferably at least 65 wt. % water.

The cosmetic carrier can moreover include 0.01 to 40 wt. %/o, preferably 0.05 to 30 wt. %, and in particular 0.1 to 20 wt. % of at least one alcohol, which can be selected from ethanol, ethyl diglycol, 1-propanol, 2-propanol, isopropanol, 1,2-propylene glycol, glycerol, diglycerol, triglycerol, 1-butanol, 2-butanol, 1,2-butanediol, 1,3-butanediol, 1-pentanol, 2-pentanol, 1,2-pentanediol, 1,5-pentanediol, 1-hexanol, 2-hexanol, 1,2-hexanediol, 1,6-hexandiol, polyethylene glycols, sorbitol, sorbitan, benzyl alcohol, phenoxyethanol or mixtures of these alcohols. The water-soluble alcohols are particularly preferred. In particular, ethanol, ethyl diglycol, 1-propanol, 2-propanol, isopropanol, 1,2-propylene glycol, glycerol, benzyl alcohol and/or phenoxyethanol and mixtures of these alcohols are preferred.

Particularly mild hair cleansing agents having excellent nourishing properties for damaged hair were able to be produced in a narrower pH range.

In a first preferred embodiment, the hair cleansing agents according to the invention thus have a pH value in the range of 4.4 to 5.0, more preferably of 4.5 to 4.9, and in particular of 4.5 to 4.8.

The suitable anionic surfactants that can be used in the hair cleansing agents according to the invention include, for example:
linear and branched fatty acids having 8 to 30 carbon atoms (soaps);
ether carboxylic acids of formula R—O—(CH₂—CH₂O)ₓ—CH₂—COOH, in which R is a linear or branched, saturated or unsaturated alkyl group having 8 to 30 carbon atoms, and x=0 or 1 to 16;
acyl sarcosides having 8 to 24 carbon atoms in the acyl group;
acyl taurides having 8 to 24 carbon atoms in the acyl group;
acyl isethionates having 8 to 24 carbon atoms in the acyl group;
sulfosuccinic acid monoalkyl and/or dialkyl esters having 8 to 24 carbon atoms in the alkyl group, and sulfosuccinic acid monoalkyl polyoxyethyl esters having 8 to 24 carbon atoms in the alkyl group and 1 to 6 oxyethyl groups;
alpha-olefin sulfonates having 8 to 24 carbon atoms;
alkyl sulfate and/or alkyl polyglycol ether sulfate salts of formula R—(O—CH₂—CH₂)ₙ—OSO₃X, in which R is preferably a linear or branched, saturated or unsaturated alkyl group having 8 to 30 carbon atoms, x denotes the number 0 or 1 to 12, and X denotes an alkali, alkaline earth, ammonium or alkanolamine ion;
sulfonates of unsaturated fatty acids having 8 to 24 carbon atoms and 1 to 6 double bonds;
esters of tartaric acid and citric acid with alcohols that represent addition products of approximately 2 to 15 molecules ethylene oxide and/or propylene oxide to fatty alcohols having 8 to 22 carbon atoms; and/or
alkyl and/or alkenyl ether phosphates of formula

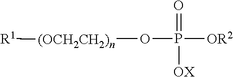

in which R¹ preferably denotes an aliphatic hydrocarbyl radical having 8 to 30 carbon atoms, R² denotes hydrogen, a (CH₂CH₂O)ₙR¹ group or X, n denotes numbers from 1 to 10, and X denotes hydrogen, an alkali metal or alkaline earth metal or the group NR³R⁴R⁵R⁶, where R³ to R⁶, independently of one another, denote a C₁ to C₄ hydrocarbyl radical.

Particularly preferred within the scope of the present invention are mild anionic surfactants that are gentle to the scalp, which within the scope of the present invention shall preferably be understood to include ether carboxylic acids, acyl isethionates, sulfosuccinic acid esters and alkyl (polyglycol ether) sulfates.

In a second preferred embodiment, the hair cleansing agents according to the invention thus preferably comprise ether carboxylic acids of formula R—O—(CH₂—CH₂O)ₓ—CH₂—COOH as anionic surfactants, in which R denotes a linear or branched, saturated or unsaturated alkyl group having 8 to 30 carbon atoms, and x=0 or 1 to 16, acyl isethionates having 8 to 24 carbon atoms in the acyl group, sulfosuccinic acid monoalkyl and/or dialkyl esters having 8 to 24 carbon atoms in the alkyl group, and sulfosuccinic acid monoalkyl polyoxyethyl esters having 8 to 24 carbon atoms in the alkyl group and 1 to 6 oxyethyl groups and/or alkyl sulfate salts and/or alkyl polyglycol ether sulfate salts of formula R—(OCH₂—CH₂)ₓ—OSO₃₋X⁺, in which R preferably denotes a linear or branched, saturated or unsaturated alkyl group having 8 to 30 carbon atoms, x denotes the number 0 or 1 to 12, and X⁺ denotes an alkali, alkaline earth, ammonium or alkanolamine ion.

Especially particularly preferred are alkyl ether sulfate salts of formula R—(OCH₂—CH₂)—OSO₃₋X⁺, in which R preferably denotes a straight-chain or branched alkyl group having 8 to 18, and in particular 10 to 16, carbon atoms, x denotes 1 to 6, and in particular 2 to 4, ethylene oxide units, and X⁺ denotes a sodium, magnesium and/or triethanolamine ion. In particular, the sodium, magnesium and/or triethanolamine salts of linear or branched lauryl, tridecyl and/or myristyl sulfates having a degree of ethoxylation of 2 to 4 are preferred, as they are commercially available, for example, under the INCI name Sodium Laureth Sulfate or Sodium Trideceth Sulfate. These surfactants have a particularly good balance between foamability and mildness.

An excessively high content of anionic surfactants is not suitable for the mild hair cleansing agents according to the invention, and for this reason these comprise 6 to 12 wt. % of one or more anionic surfactants, based on the weight of the hair cleansing agents. In particular, the hair cleansing agents according to the invention preferably comprise alkyl sulfate salts and/or alkyl polyglycol ether sulfate salts of the aforementioned formula in amounts of 6 to 12 wt. %.

A further preferred embodiment of the invention is thus characterized in that the hair cleansing agents, based on the total weight thereof, comprise 6 to 12 wt. %, preferably 7 to 11 wt %, and in particular 8 to 10 wt. % of at least one anionic alkyl ether sulfate salt of formula R—(OCH₂—CH₂)ₓ—OSO₃₋X⁺, in which R denotes a straight-chain or branched alkyl group having 8 to 18, and in particular 10 to 16 carbon atoms, x denotes 1 to 6, and in particular 2 to 4 ethylene oxide units, and X⁺ denotes a sodium, magnesium and/or triethanolamine ion.

So as to achieve optimal foaming properties and mildness at the same time, the hair cleansing agents according to the invention comprise 0.1 to 5 wt. % of at least one non-ionic surfactant, in addition to at least one anionic surfactant, based on the total weight of the agents.

The non-ionic surfactants/emulsifiers that are suitable according to the invention preferably include:
C₈ to C₃₀ fatty acid monoesters and diesters of addition products of 1 to 30 moles ethylene oxide to glycerol;
amine oxides;
addition products of 2 to 50 moles ethylene oxide and/or 0 to 5 moles propylene oxide to linear and branched fatty alcohols having 8 to 30 carbon atoms, to fatty acids having 8 to 30 carbon atoms, and to alkyl phenols having 8 to 15 carbon atoms in the alkyl group;
sorbitan fatty acid esters and addition products of ethylene oxide to sorbitan fatty acid esters, such as polysorbates;
sugar fatty acid esters and addition products of ethylene oxide to sugar fatty acid esters;
fatty acid alkanolamides of the general formula

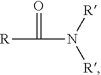

in which R preferably denotes a linear or branched, saturated or unsaturated alkyl or alkenyl group having 8 to 24 carbon atoms, and the groups R¹ denote hydrogen or the group —(CH₂)ₙOH, in which n denotes the number 2 or 3, with the proviso that at least one of the groups R′ denotes the aforementioned group —(CH₂)ₙOH;
addition products of ethylene oxide to fatty amines and/or
alkyl oligoglucosides, and in particular alkyl oligoglucosides based on hydrogenated C_{12/14} coconut alcohol having a DP of 1 to 3, as they are commercially available, for example, under the INCI name “Coco-Glucoside,” “Decyl Glucoside” and/or “Lauryl Glucoside.”

The use of high-foaming and/or foam-stabilizing non-ionic, mild surfactants has proven to be extremely satisfactory, and for this reason, hair cleansing agents according to the invention in a third particularly preferred embodiment are characterized by comprising at least one non-ionic surfactant, selected from C₈ to C₃₀ fatty acid monoesters and/or diesters of addition products of 1 to 30 moles ethylene oxide to glycerol, addition products of 2 to 50 moles ethylene oxide and/or 0 to 5 moles propylene oxide to linear and branched fatty alcohols having 8 to 30 carbon atoms, fatty acid alkanolamides of the general formula

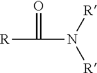

in which R preferably denotes a linear or branched, saturated or unsaturated alkyl or alkenyl group having 8 to 24 carbon atoms, and the groups R′ denote hydrogen or the group —(CH₂)ₙOH, in which n denotes the number 2 or 3, with the proviso that at least one of the groups R′ denotes the aforementioned group —(CH₂)ₙOH, and/or alkyl polyglucosides.

In addition to the mildness, fatty acid alkanolamides have foam-stabilizing, thickening and nourishing (lipid-replenishing) properties, and for this reason, hair cleansing agents according to the invention in a further advantageous embodiment that are particularly preferred are those that, based on the total weight thereof, comprise 0.1 to 5 wt. %, preferably 0.2 to 3 wt. %, and in particular 0.4 to 2 wt. % of at least one fatty acid alkanolamide of the aforementioned formula. In particular, a fatty acid alkanolamide known under the INCI name Cocamide MEA is preferred.

In addition to the surfactant mixture of anionic and non-ionic surfactants in specific quantities, the hair cleansing agents according to the invention comprise at least one cationic nourishing polymer in an amount of 0.1 to 2 wt. % (based on the total weight of the hair cleansing agents) so as to enhance the nourishing properties of damaged hair.

Suitable cationic nourishing polymers shall be understood to mean, for example:
quaternized cellulose polymers, in particular Polyquaternium-10, as they are commercially available under the designations Celquat® and Polymer JR®;
hydrophobically modified cellulose derivatives, for example the cationic polymers sold under the trade name SoftCat®;
cationic alkyl polyglycosides;
cationized honey, for example the commercial product Honeyquat® 50;
cationic guar derivatives, such as in particular the products sold under the trade names Cosmedia®Guar N-Hance® and Jaguar®;
polymeric dimethyl diallyl ammonium salts and the copolymers thereof with esters and amides of acrylic acid and methacrylic acid, especially Polyquatemium-6 and Polyquaternium-7. The products available commercially under the designations Merquat®100 (poly(dimethyldiallylammonium chloride)) and Merquat®550 (dimethyldiallylammonium chloride/acrylamide copolymer) are examples of such cationic polymers;
copolymers of vinylpyrrolidone with quaternized derivatives of dialkyl aminoalkyl acrylate and methacrylate, such as diethyl sulfate-quatemized vinylpyrrolidone/dimethylaminoethyl methacrylate copolymers. Such compounds are commercially available under the designations Gafquat®734 and Gafquat®755;
vinylpyrrolidone/vinylimidazolium methochloride copolymers, as they are offered under the designations Luviquat® FC 370, FC 550, FC 905 and HM 552;
quatemized polyvinyl alcohol;
and the polymers known by the designations
Polyquaternium 2, Polyquaternium 17, Polyquatemium 18, Polyquaternium-24, Polyquaternium 27, Polyquatemium-32, Polyquatemium-37, Polyquatemium 74, and Polyquaternium 89.

Particularly preferred cationic polymers are quaternized cellulose polymers, hydrophobically modified quaternized cellulose polymers, cationic guar derivatives and/or cationic polymers based on acrylic acid (derivatives), which particularly preferably are selected from the polymers known by the INCI names Guar Hydroxypropyltrimonium Chloride, Polyquaternium-6, Polyquatemium-7, Polyquatemium-10, Polyquaternium-37 and/or Polyquaternium-67.

In a fourth preferred embodiment, hair cleansing agents according to the invention thus comprise at least one quaternized cellulose polymer, a hydrophobically modified quaternized cellulose polymer, a cationic guar derivative, a polymeric dimethyl diallyl ammonium salt and the copolymers thereof with esters and/or amides of acrylic acid and methacrylic acid, a copolymer of vinylpyrrolidone with quaternized derivatives of dialkylaminoalkyl acrylate and methacrylate and/or polyvinylpyrrolidone as the cationic nourishing polymer.

Cationic polysaccharide polymers, and in particular quaternized cellulose polymers, have been found to be especially particularly preferred cationic nourishing polymers for the hair cleansing agents according to the invention.

In a further preferred embodiment, hair cleansing agents according to the invention, based on the total weight thereof, thus comprise 0.01 to 2 wt. %, preferably 0.1 to 1.5 wt. %, and in particular 0.2 to 1 wt. % of at least one quaternized cellulose polymer, and in particular of a cationic polymer obtainable by reacting hydroxyethyl cellulose with trimethylammonium-substituted epoxides and known under the INCI name Polyquaternium-10.

The above-described excellent balance between cleansing, mild and nourishing properties of the hair cleansing agents according to the invention was not achieved until calcium lactate (or alternatively a mixture of lactic acid and an inorganic calcium salt) was added to the mixture of anionic surfactants, non-ionic surfactants and cationic nourishing polymers on the one hand, and the pH value was set to a value in the range of 4.3 to 5.0 on the other hand. It is thus advantageous if no further acids than lactic acid and/or calcium lactate are used to set the pH value.

To achieve the described nourishing effects, it is irrelevant whether calcium lactate or a mixture of lactic acid and an inorganic calcium salt is used in the hair cleansing agents. However, in the selection of the inorganic calcium salts, calcium chloride, calcium hydroxide, calcium sulfate and/or calcium oxide have proven to be particularly suitable.

Likewise, it was found that calcium lactate or the mixture of lactic acid and an inorganic calcium salt must be used in a particular amount in the hair cleansing agents to achieve optimal nourishing effects. For example, particularly good nourishing effects (detanglability, combability and anti-frizz effect) were achieved with hair cleansing agents that, based on the total weight thereof, comprise
i. 0.01 to 3 wt. %, preferably 0.02 to 2.5 wt. % and in particular 0.05 to 2 wt. % calcium lactate or
ii. 0.1 to 5 wt. %, preferably 0.5 to 4 wt. %, and in particular 1 to 3 wt. % lactic acid and 0.05 to 3 wt. %, preferably 0.1 to 2 wt. % and in particular 0.2 to 1 wt. % calcium hydroxide.

In a fifth preferred embodiment, hair cleansing agents according to the invention thus comprise calcium chloride, calcium hydroxide, calcium sulfate and/or calcium oxide as the inorganic calcium salt d).

In a sixth preferred embodiment, hair cleansing agents according to the invention, based on the total weight thereof, thus comprise
i. 0.01 to 3 wt. %, preferably 0.02 to 2.5 wt. % and in particular 0.05 to 2 wt. % calcium lactate or
ii. 0 1 to 5 wt. %, preferably 0.5 to 4 wt. %, and in particular 1 to 3 wt. % lactic acid and 0.05 to 3 wt. %, preferably 0.1 to 2 wt. % and in particular 0.2 to 1 wt. % calcium hydroxide.

An especially particularly preferred embodiment of the present invention is characterized in that the hair cleansing agents, based on the total weight thereof, comprise
a) 6 to 12 wt. % of at least one anionic alkyl ether sulfate salt of formula R—(OCH₂—CH₂)ₓ—OSO₃₋X+, in which R denotes a straight-chain or branched alkyl group having 8 to 18, and in particular 10 to 16, carbon atoms, x denotes 1 to 6, and in particular 2 to 4 ethylene oxide units, and X⁺ denotes a sodium, magnesium and/or triethanolamine cation,
b) 0.1 to 5 wt. % of at least one fatty acid alkanolamide of formula

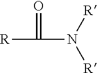

in which R preferably denotes a linear or branched, saturated or unsaturated alkyl or alkenyl group having 8 to 24 carbon atoms, and the groups R′ denote hydrogen or the group —(CH₂)ₙOH, in which n denotes the number 2 or 3, with the proviso that at least one of the groups R′ denotes the aforementioned group —(CH₂)ₙOH;
c) 0.01 to 2 wt. % of at least one quaternized cellulose polymer, and
d) (i) 0.01 to 3 wt. % calcium lactate or
   (ii) 0.1 to 5 wt. % lactic acid and 0.05 to 3 wt. % calcium hydroxide, and have a pH value in the range of 4.3 to 5.0, preferably of 4.4 to 5.0, preferably of 4.5 to 4.9, and in particular of 4.5 to 4.8.

Within this embodiment, in particular hair cleansing agents are preferred that, based on the total weight thereof, comprise
a) 1 to 11 wt. % of at least one sodium, magnesium and/or triethanolamine salt of a linear or branched lauryl, tridecyl and/or myristyl sulfate having a degree of ethoxylation of 2 to 4,
b) 0.2 to 3 wt. % of at least one fatty acid alkanolamide known under the INCI name Cocamide MEA,
c) 0.1 to 1.5 wt. % of at least one quaternized cellulose polymer known under the INCI name Polyquaternium-10, and
d) (i) 0.02 to 2.5 wt. % calcium lactate or
   (ii) 0.5 to 4 wt. % lactic acid and 0.1 to 2 wt. % calcium hydroxide, and have a pH value in the range of 4.5 to 4.9, and in particular of 4.5 to 4.8.

The visual and haptic properties of the hair cleansing agents according to the invention can be even further increased when other hair nourishing agents are added to these. Possible other hair nourishing agents that are suitable are cosmetically suitable oil, fat and/or wax components, vitamins and/or protein hydrolysates.

Cosmetically suitable oil, wax and/or fat components can preferably be selected from mineral, natural and synthetic oil components and/or fats.

Usually, triglycerides and mixtures of triglycerides are used as natural (vegetable) oils. Preferred natural oils are coconut oil, (sweet) almond oil, walnut oil, peach kernel oil, apricot kernel oil, avocado oil, tea tree oil, soy bean oil, sesame oil, sunflower oil, tsubaki oil, evening primrose oil, rice bran oil, palm kernel oil, mango kernel oil, lady's smock oil, thistle oil, macadamia nut oil, grape seed oil, amaranth seed oil, argan oil, bamboo oil, olive oil, wheat germ oil, pumpkin seed oil, mallow oil, hazelnut oil, safflower oil, canola oil, sasanqua oil, jojoba oil, rambutan oil, cocoa butter and/or shea butter.

Mineral oils that are used are in particular petroleum, paraffin and isoparaffin oils, and synthetic hydrocarbons. One example of a hydrocarbon that can be used, for example, is 1,3-di-(2-ethylhexyl)cyclohexane (Cetiol® S), which is available as a commercial product.

A dialkyl ether can furthermore serve as the oil component. Suitable dialkyl ethers are in particular di-n-alkyl ethers having in total between 12 and 36 carbon atoms, in particular 12 to 24 carbon atoms, such as di-n-octyl ether, di-n-decyl ether, di-n-nonyl ether, di-n-undecyl ether, di-n-dodecyl ether, n-hexyl-n-octyl ether, n-octyl-n-decyl ether, n-decyl-n-undecyl ether, n-undecyl-n-dodecyl ether and n-hexyl-n-undecyl ether, and di-tert-butyl ether, di-iso-pentyl ether, di-3-ethyldecyl ether, tert-butyl-n-octyl ether, iso-pentyl-n-octyl ether and 2-methyl-pentyl-n-octyl ether. The di-n-octyl ether that is commercially available under the designation Cetiol® OE is particularly preferred.

Silicone compounds are preferably used as synthetic oils. Silicones effectuate outstanding conditioning properties on the hair. In particular, they positively affect the feel of the hair and the softness of the hair in many instances. It is therefore desirable to use silicones in cosmetic hair treatment products. Suitable silicones can be selected from:
(i) polyalkylsiloxanes, polyarylsiloxanes, polyalkylarylsiloxanes, which are volatile or non-volatile, straight-chain, branched or cyclic, cross-linked or not cross-linked;
(ii) polysiloxanes containing, in the general structure thereof, one or more organofunctional groups, which are selected from:
   a) substituted or unsubstituted aminated groups;
   b) (per)fluorinated groups;
   c) thiol groups;
   d) carboxylate groups;
   e) hydroxylated groups;
   f) alkoxylated groups;
   g) acyloxyalkyl groups;
   h) amphoteric groups;
   i) bisulfite groups;
   j) hydroxyacylamino groups;
   k) carboxy groups;
   I) sulfonic acid groups; and
   m) sulfate or thiosulfate groups;
   (iii) linear polysiloxane(A)/polyoxyalkylene(B) block copolymers of the (A-B)ₙ type where n >3;
   (iv) grafted silicone polymers having a non-silicone-containing, organic skeleton, which are composed of an organic main chain formed of organic monomers that do not include any silicone, onto which at least one polysiloxane macromer was grafted in the chain and optionally on at least one chain end;
   (v) grafted silicone polymers having a polysiloxane skeleton onto which non-silicone-containing, organic monomers were grafted that include a polysiloxane main chain, onto which at least one organic macromer that does not include silicone was grafted in the chain and optionally on at least one of the ends thereof;
   (vi) or the mixtures thereof.

Fats shall be understood to mean fatty acids, fatty alcohols and natural and synthetic waxes, which may be present either in solid form or liquid in an aqueous dispersion. Linear and/or branched, saturated and/or unsaturated fatty acids having 6 to 30 carbon atoms can be used as fatty acids. Fatty acids having 10 to 22 carbon atoms are preferred. Among these, for example, isostearic acids should be mentioned, such as the commercial products Emersol® 871 and Emersol® 875, and isopalmitic acids, such as the commercial product Edenor® IP 95, and all further fatty acids sold under the trade name Edenor® (Cognis). Further typical examples of such fatty acids are caproic acid, caprylic acid, 2-ethylhexanoic acid, caprinic acid, lauric acid, isotridecanoic acid, myristic acid, palmitic acid, palmitoleic acid, stearic acid, isostearic acid, oleic acid, elaidic acid, petroselinic acid, linoleic acid, linolenic acid, eleostearic acid, arachidic acid, gadoleic acid, behenic acid and erucic acid, and the technical mixtures thereof.

Usually, the fatty acid cuts that are obtainable from coconut oil or palm oil are particularly preferred; in particular, the use of stearic acid is generally preferred. Suitable fatty alcohols are saturated, monounsaturated or polyunsaturated, branched or unbranched fatty alcohols having C₆ to C₃₀, preferably C₁₀ to C₂₂, and especially particularly preferably C₁₂ to C₂₂ carbon atoms. It is possible to use, for example, decanol, octanol, octenol, dodecenol, decenol, octadienol, dodecadienol, decadienol, oleyl alcohol, eruca alcohol, ricinol alcohol, stearyl alcohol, isostearyl alcohol, cetyl alcohol, lauryl alcohol, myristyl alcohol, arachidyl alcohol, capryl alcohol, capric alcohol, linoleyl alcohol, linolenyl alcohol and behenyl alcohol, and the guerbet alcohols thereof, wherein this enumeration is to be of an exemplary and non-limiting nature. The fatty alcohols, however, are preferably derived from natural fatty acids, wherein usually a production from the esters of fatty acids by way of reduction can be assumed. According to the invention, it is likewise possible to use fatty alcohol cuts that are produced by reducing naturally occurring triglycerides, such as suet, palm oil, peanut oil, rapeseed oil, cottonseed oil, soy bean oil, sunflower oil, and linseed oil, or from the transesterification products thereof with appropriate alcohols, and thus represent a mixture of different fatty alcohols. Such substances are available for purchase, for example, under the designations Stenol®, such as Stenol® 1618, or Lanette®, such as Lanette®, or Lorol®, such as Lorol® C8, Lorol® C14, Lorol® C18, Lorol® C8-18, HD-Ocenol®, Crodacol®, such as Crodacol® CS, Novol®, Eutanol® G, Guerbitol® 16, Guerbitol® 18, Guerbitol® 20, Isofol® 12, Isofol® 16, Isofol® 24, Isofol® 36, Isocarb® 12, Isocarb® 16 or Isocarb® 24. It is also possible according to the invention, of course, to use wool wax alcohols, as they are available for purchase, for example, under the designations Corona®, White Swan®, Coronet® or Fluilan®.

Suitable natural or synthetic waxes that can be used are solid paraffins or isoparaffins, carnauba waxes, beeswaxes, candelilla waxes, ozokerites, ceresin, spermaceti wax, sunflower wax, fruit waxes such as apple wax or citrus wax, and microwaxes made of PE or PP. Such waxes are available from Kahl & Co., Trittau, for example.

Further fats are, for example
ester oils. Ester oils shall be understood to mean the esters of C₆ to C₃₀ fatty acids having C₂ to C₃₀ fatty alcohols. The monoesters of fatty acids with alcohols having 2 to 24 carbon atoms are preferred. Examples of fatty acid components used in the esters are caproic acid, caprylic acid, 2-ethylhexanoic acid, caprinic acid, lauric acid, isotridecanoic acid, myristic acid, palmitic acid, palmitoleic acid, stearic acid, isostearic acid, oleic acid, elaidic acid, petroselinic acid, linoleic acid, linolenic acid, eleostearic acid, arachidic acid, gadoleic acid, behenic acid and erucic acid, and the technical mixtures thereof.
Examples of the fatty alcohol components in the ester oils are isopropyl alcohol, caproic alcohol, caprylic alcohol, 2-ethylhexyl alcohol, capric alcohol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinyl alcohol, linoleyl alcohol, linolenyl alcohol, eleostearyl alcohol, arachyl alcohol, gadoleyl alcohol, behenyl alcohol, erucyl alcohol, and brassidyl alcohol, and the technical mixtures thereof Particularly preferred are isopropyl myristate (Rilanit® IPM), isononanoic acid-C₁₆₋₁₈ alkyl ester (Cetiol® SN), 2-ethylhexyl palmitate (Cegesoft® 24), stearic acid-2-ethylhexyl ester (Cetiol® 868), cetyl oleate, glycerin tricaprylate, coconut fatty alcohol caprinate/caprylate (Cetiol® LC), n-butyl stearate, oleyl erucate (Cetiol® J 600), isopropyl palmitate (Rilanit® IPP), oleyl oleate (Cetiol®), lauric acid hexyl ester (Cetiol® A), di-n-butyl adipate (Cetiol® B), myristyl myristate (Cetiol® MM), cetearyl isononanoate (Cetiol® SN), oleic acid decyl ester (Cetiol® V).
dicarboxylic acid esters such as di-n-butyl adipate, di-(2-ethylhexyl) adipate, di-(2-ethylhexyl) succinate and diisotridecyl acelaate, and diol esters such as ethylene glycol dioleate, ethylene glycol diisotridecanoate, propylene glycol di(2-ethylhexanoate), propylene glycol diisostearate, propylene glycol dipelargonate, butanediol diisostearate, neopentyl glycol dicaprylate;
symmetrical, asymmetrical or cyclic esters of carbonic acid with fatty alcohols;
glycerol carbonate or dicaprylyl carbonate (Cetiol® CC);
ethoxylated and/or non-ethoxylated mono-, di- and tri-fatty acid esters of saturated and/or unsaturated linear and/or branched fatty acids with glycerol, such as Monomuls®90-O18, Monomuls®90-L12, Cetiol® HE or Cutina® MD.

The percentage by weight of the oil, wax and/or fat components based the total weight of the hair cleansing agents according to the invention is preferably 0.01 to 5 wt. %, particularly preferably 0.025 to 4 wt. %, and in particular 0.05 to 3 wt. %.

So as to increase the luster of the hair and achieve a softer feel of the hair, it has proven to be particularly preferred if the hair cleansing agents according to the invention furthermore comprise at least one silicone and/or at least one vegetable oil as the oil, fat and/or wax component.

A further preferred embodiment of the invention is thus characterized by additionally comprising, based on the total weight thereof, 0.1 to 5 wt. % of at least one silicone and/or at least one vegetable oil.

Suitable protein hydrolysates shall be understood to mean product mixtures that can be obtained by the acidically, basically or enzymatically catalyzed degradation of proteins. Protein hydrolysates of plant, animal and/or marine origin can be used.

Animal protein hydrolysates are, for example, elastin, collagen, keratin, silk and milk protein hydrolysates, which can also be present in the form of salts. Such products are sold, for example, under the trademarks Dehylan® (Cognis), Promois® (Interorgana), Collapuron® (Cognis), Nutrilan® (Cognis), Gelita-Sol® (Deutsche Gelatine Fabriken Stoess & Co), Lexein® (Inolex), and Kerasol® (Croda).

Protein hydrolysates of plant origin, such as soy, almond, rice, pea, potato and wheat protein hydrolysates, are preferred. Such products are available, for example, under the trademarks Gluadin® (Cognis), DiaMin® (Diamalt), Lexein® (Inolex), and Crotein®(Croda).

It is also possible to use cationized protein hydrolysates, wherein the underlying protein hydrolysate can stem from animals, for example from collagen, milk or keratin, from plants, for example from wheat, maize, rice, potatoes, soy or almonds, from marine life forms, for example from fish collagen or algae, or from protein hydrolysates obtained by way of biotechnology. The protein hydrolysates underlying the cationic derivatives can be obtained from the corresponding proteins by way of a chemical, in particular alkaline or acid hydrolysis, an enzymatic hydrolysis and/or a combination of the two types of hydrolysis. The hydrolysis of proteins generally results in a protein hydrolysate having a molecular weight distribution from approximately 100 daltons to several thousand daltons Cationic protein hydrolysates in which the underlying protein component has a molecular weight of 100 to 25,000 daltons, and preferably 250 to 5000 daltons, are preferred. Moreover, cationic protein hydrolysates shall be understood to mean quaternized amino acids and the mixtures thereof. The quaternization of the protein hydrolysates or of the amino acids is frequently carried out using quaternary ammonium salts, such as N,N-dimethyl-N-(n-alkyl)-N-(2-hydroxy-3-chloro-n-propyl) ammonium halides. The cationic protein hydrolysates can moreover also be further derivatized. Typical examples of the cationic protein hydrolysates and derivatives that shall be mentioned are the commercially available products known under the following INCI names: Cocodimonium Hydroxypropyl Hydrolyzed Collagen, Cocodimonium Hydroxypropyl Hydrolyzed Casein, Cocodimonium Hydroxypropyl Hydrolyzed Collagen, Cocodimonium Hydroxypropyl Hydrolyzed Hair Keratin, Cocodimonium Hydroxypropyl Hydrolyzed Keratin, Cocodimonium Hydroxypropyl Hydrolyzed Rice Protein, Cocodimonium Hydroxypropyl Hydrolyzed Silk, Cocodimonium Hydroxypropyl Hydrolyzed Soy Protein, Cocodimonium Hydroxypropyl Hydrolyzed Wheat Protein, Cocodimonium Hydroxypropyl Silk Amino Acids, Hydroxypropyl Arginine Lauryl/Myristyl Ether HCl, Hydroxypropyltrimonium Gelatin, Hydroxypropyltrimonium Hydrolyzed Casein, Hydroxypropyltrimonium Hydrolyzed Collagen, Hydroxypropyltrimonium Hydrolyzed Conchiolin Protein, Hydroxypropyltrimonium Hydrolyzed keratin, Hydroxypropyltrimonium Hydrolyzed Rice Bran Protein, Hydroxypropyltrimonium Hydrolyzed Silk, Hydroxypropyltrimonium Hydrolyzed Soy Protein, Hydroxypropyl Hydrolyzed Vegetable Protein, Hydroxypropyltrimonium Hydrolyzed Wheat Protein, Hydroxypropyltrimonium Hydrolyzed Wheat Protein/Siloxysilicate, Laurdimonium Hydroxypropyl Hydrolyzed Soy Protein, Laurdimonium Hydroxypropyl Hydrolyzed Wheat Protein, Laurdimonium Hydroxypropyl Hydrolyzed Wheat Protein/Siloxysilicate, Lauryldimonium Hydroxypropyl Hydrolyzed Casein, Lauryldimonium Hydroxypropyl Hydrolyzed Collagen, Lauryldimonium Hydroxypropyl Hydrolyzed Keratin, Lauryldimonium Hydroxypropyl Hydrolyzed Silk, Lauryldimonium Hydroxypropyl Hydrolyzed Soy Protein, Steardimonium Hydroxypropyl Hydrolyzed Casein, Steardimonium Hydroxypropyl Hydrolyzed Collagen, Steardimonium Hydroxypropyl Hydrolyzed Keratin, Steardimonium Hydroxypropyl Hydrolyzed Rice Protein, Steardimonium Hydroxypropyl Hydrolyzed Silk, Steardimonium Hydroxypropyl Hydrolyzed Soy Protein, Steardimonium Hydroxypropyl Hydrolyzed Vegetable Protein, Steardimonium Hydroxypropyl Hydrolyzed Wheat Protein, Steartrimonium Hydroxyethyl Hydrolyzed Collagen, Quaternium-76 Hydrolyzed Collagen, Quaternium-79 Hydrolyzed Collagen, Quaternium-79 Hydrolyzed Keratin, Quaternium-79 Hydrolyzed Milk Protein, Quaternium-79 Hydrolyzed Silk, Quaternium-79 Hydrolyzed Soy Protein, Quaternium-79 Hydrolyzed Wheat Protein.

The percentage by weight of the protein hydrolysate or protein hydrolysates based the total weight of the hair treatment agents according to the invention is preferably 0.001 to 5 wt. %, more preferably 0.025 to 3 wt. %, and in particular 0.05 to 2 wt. %.

Suitable vitamins shall preferably be understood to mean the following vitamins, provitamins, and vitamin precursors, and the derivatives thereof:

Vitamin A: The group of substances referred to as vitamin A includes retinol (vitamin A₁) and 3,4-didehydroretinol (vitamin A₂). β-carotene is the provitamin of retinol. Possible vitamin A components are, for example, vitamin A acid and the esters thereof, vitamin A aldehyde, and vitamin A alcohol and the esters thereof, such as palmitate and acetate.

Vitamin B: The vitamin B group or vitamin B complex includes, among other things,
Vitamin B₁ (thiamine)
Vitamin B₂ (riboflavin)
Vitamin B₃. The compounds nicotinic acid and nicotinic acid amide (niacinamide) are often covered by this designation.
Vitamin B₅ (pantothenic acid and panthenol). Within this group, panthenol is preferably used. Derivatives of panthenol that can be used are in particular the esters and ethers of panthenol, pantolactone and cationically derivatized panthenols. Individual representatives are, for example, panthenol triacetate, panthenol monoethyl ether and the monoacetate thereof, and cationic panthenol derivatives.
Vitamin B₆ (pyridoxine, pyridoxamine and pyridoxal).

Vitamin C (ascorbic acid): Use in the form of the palmitic acid ester, glucosides or phosphates can be preferred. Use in combination with tocopherols can likewise be preferred.

Vitamin E (tocopherols, in particular α-tocopherol).

Vitamin F: The term “Vitamin F” is usually understood to mean essential fatty acids, in particular linoleic acid, linolenic acid and arachidonic acid.

Vitamin H: Vitamin H refers to the compound (3aS,4S,6aR)-2-oxohexahydrothienol[3,4-d]-imidazole-4-valeric acid, although this has since become established by the trivial name biotin.

Vitamins, provitamins and vitamin precursors from the groups A, B, E and H are particularly preferred. Nicotinic acid amide, biotin, pantolactone and/or panthenol are especially particularly preferred. In particular, panthenol is preferred.

The percentage by weight of the vitamin(s), vitamin derivative(s) and/or vitamin precursor(s) based the total weight of the cosmetic agents is preferably 0.001 to 2 wt. %, particularly preferably 0.005 to 1 wt. %, and in particular 0.01 to 0.5 wt. %.

A further preferred embodiment of the invention is characterized by additionally comprising, based on the total weight thereof, 0.001 to 2 wt. % of at least one vitamin, vitamin derivative and/or vitamin salt and/or 0.001 to 5 wt. % of at least one, optionally cationized, protein hydrolysate.

It may furthermore be advantageous to also add a UV filter to the hair cleansing agents according to the invention so as to protect in particular already damaged hair from harmful UV radiation exposure.

In a further preferred embodiment, hair cleansing agents according to the invention, based on the total weight thereof, thus furthermore comprise 0.01 to 0.5 wt. % of at least one UV filter.

Suitable UV filters are not subject to any general restrictions with regard to the structure and the physical properties thereof. Rather, all UV filters that can be used in the cosmetics field, having an absorption maximum in the UVA (315-400 nm) region, in the UVB (280-315 nm) region or in the UVC (<280 nm) region, are suitable. UV filters having an absorption maximum in the UVB region, in particular in the range from approximately 280 to approximately 300 nm, are particularly preferred.

The UV filters can be selected, for example, from substituted benzophenones, p-aminobenzoic acid esters, diphenylacrylic acid esters, cinnamic acid esters, salicylic acid esters, benzimidazoles and o-aminobenzoic acid esters.

Especially water-soluble UV filters are suitable for producing and stabilizing the hair cleansing agents according to the invention, such as the UV filter known under the INCI name Benzophenone-4.

In addition to the above-mentioned active ingredients and carrier substances, the hair cleansing agents according to the invention can include a number of other ingredients that impart advantageous properties to these. The preferred optional active ingredients that can be used in the agents according to the invention include, for example:
amphoteric and/or zwitterionic surfactants, which can preferably be used in the hair cleansing agents, based on the total weight thereof, in an amount of 0.5 to 20 wt. %, more preferably 1 to 15 wt. %, particularly preferably 1.25 to 10 wt. %, and in particular 1.5 to 7.5 wt. %; and
anti-dandruff active ingredients, which can preferably be used in the hair cleansing agents, based on the total weight thereof, in an amount of 0.01 to 7.5 wt. %, particularly preferably 0.05 to 5 wt. %, and in particular of 0.1 to 3 wt. %.

The suitable amphoteric and/or zwitterionic surfactants that can be used in the hair cleansing agents according to the invention include, for example, one or more compounds of the following formulas (I) to (VII), in which the group R denotes a straight-chain or branched, saturated or monounsaturated or polyunsaturated alkyl or alkenyl group having 7 to 23 carbon atoms (formulas (I) and (II)) or a straight-chain or branched, saturated or monounsaturated or polyunsaturated alkyl or alkenyl group having 8 to 24 carbon atoms (formulas (III) to (VII)):

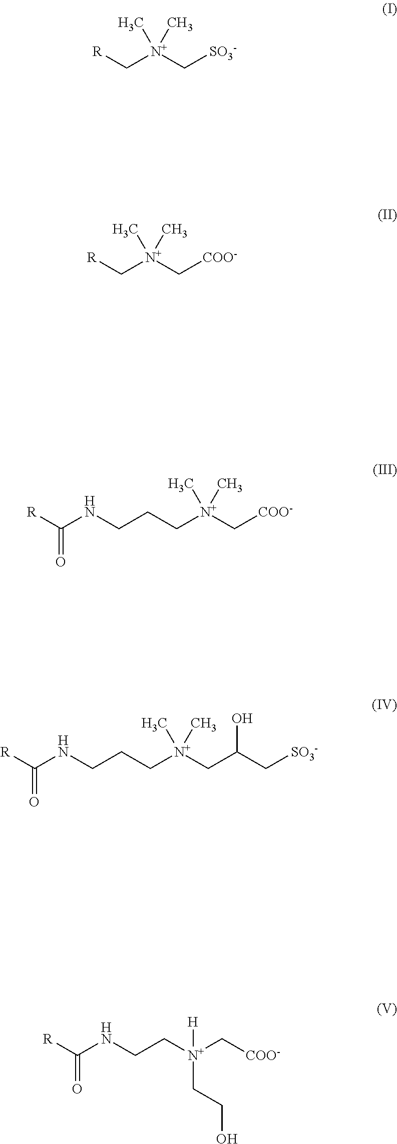

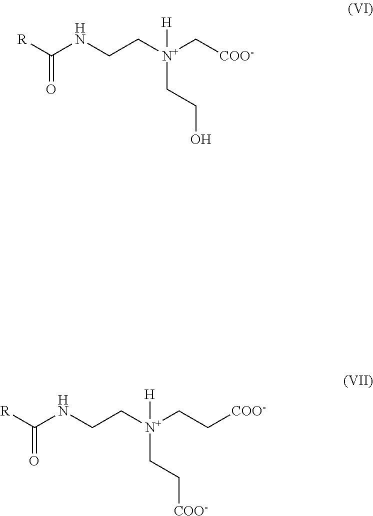

Preferred amphoteric and/or zwitterionic surfactants of one of the aforementioned formulas (I) to (VII) predominantly include a straight-chain or branched, saturated, monounsaturated or polyunsaturated alkyl group having 8 to 20, more preferably 8 to 18, and in particular 8 to 16 carbon atoms as the group R. Amphoteric and/or zwitterionic surfactants in which the group R is derived from coconut fat are particularly preferred. The amphoteric/zwitterionic surfactants known under the INCI names Sodium Cocoamphoacetate, Disodium Cocoamphodiacetate, Sodium Lauroamphoacetate, Sodium Lauroamphodiacetate, Sodium Cocoamphopropionate, Disodium Cocoamphodipropionate, Coco Betaine, Lauryl Betaine Cocamidopropylbetain and/or Lauramidopropylbetain and commercially available from several providers are especially particularly preferred. In particular, surfactants by the INCI names Cocoampho(di)acetate and/or Cocamidopropylbetain are preferred.

Suitable active anti-dandruff ingredients can be selected from piroctone olamine, climbazole, zinc pyrithione, ketoconazoles, salicylic acid, sulfur, selenium sulfide, tar preparations, undecenoic acid derivatives, burdock root extracts, poplar extracts, stinging nettle extracts, walnut shell extracts, birch extracts, willow bark extracts, rosemary extracts and/or arnica extracts. Climbazole, zinc pyrithione and piroctone olamine are particularly preferred, and in particular zinc pyrithione is preferred.

The further optional components that can be used in the hair cleansing agents according to the invention include, for example:
plant extracts and/or
humectants.

Suitable plant extracts shall be understood to mean extracts that can be produced from all parts of a plant. These extracts are usually produced by extracting the entire plant.

However, in isolated cases, it may also be preferred to produce the extracts exclusively from flowers and/or leaves of the plant. Especially suited are the extracts of green tea, oak bark, stinging nettle, witch hazel, hops, chamomile, burdock root, horsetail, hawthorn, lime blossom, lychee, almond, aloe vera, pine needle, horse chestnut, sandalwood, juniper, coconut, mango, apricot, lime, wheat, kiwi, melon, orange, grapefruit, sage, rosemary, birch, mallow, lady's smock, creeping thyme, yarrow, thyme, balm, restharrow, coltsfoot, marshmallow, ginseng, ginger root, Echinacea purpurea, Olea europea, Boerhavia diffusa roots, Foeniculum vulgare, and Apium graveolens. Particularly preferred for use in the compositions according to the invention are the extracts of green tea, stinging nettle, witch hazel, chamomile, aloe vera, ginseng, Echinacea purpurea, Olea europea, and Boerhavia diffusa roots.

Extracting agents for producing the described plant extracts that may be used are water, alcohols and the mixtures thereof. Among the alcohols, lower alcohols, such as ethanol and isopropanol, in particular, however, polyhydric alcohols, such as ethylene glycol and propylene glycol, both as the sole extracting agent and also in a mixture with water, are preferred. Plant extracts based on water/propylene glycol at a ratio of 1:10 to 10:1 have proven to be particularly suitable. The plant extracts can be used either in pure form or in dilute form. If they are used in dilute form, they usually include approximately 2 to 80 wt. % active substance and, as solvent, the extracting agent or extracting agent mixture used during extraction thereof. The plant extracts may preferably be used in the hair cleansing agents according to the invention, based on the total weight of the agents, in an amount of 0.01 to 10 wt. %, more preferably of 0.05 to 7.5 wt. %, and in particular of 0.1 to 5 wt. %.

Suitable humectants or auxiliary penetrating agents and/or swelling agents that can be added to the hair cleansing agents according to the invention include, for example, urea and urea derivatives, guanidine and the derivatives thereof, arginine and the derivatives thereof, waterglass, imidazole and the derivatives thereof, histidine and the derivatives thereof, benzyl alcohol, glycerol, glycol and glycol ethers, propylene glycol and propylene glycol ethers, for example propylene glycol monoethyl ethers, carbonates, hydrogen carbonates, diols and triols, and, in particular 1,2-diols and 1,3-diols, such as 1,2-propanediol, 1,2-pentanediol, 1,2-hexanediol, 1,2-dodecanediol, 1,3-propanediol, 1,6-hexanediol, 1,5-pentanediol, and 1,4-butanediol. The humectants may preferably be used in the hair cleansing agents according to the invention, based on the total weight thereof, in amounts of 0.01 to 10 wt. %, more preferably in amounts of 0.05 to 5 wt. %, and in particular in amounts of 0.1 to 3 wt. %.

Further active ingredients, auxiliary substances and additives that can be used in the hair cleansing agents according to the invention are, for example:
thickening agents such as gelatin or gums, for example agar-agar, guar gum, alginates, xanthan gum, gum arabic, karaya gum, locust bean gum, linseed gums, dextrans, cellulose derivatives, for example methyl cellulose, hydroxyalkyl cellulose and carboxymethyl cellulose, starch fractions and derivatives, such as amylose, amylopectin and dextrins, clays and phyllosilicates, such as bentonite or fully synthetic hydrocolloids, such as polyvinyl alcohol, the Ca, Mg or Zn soaps;
structurants such as maleic acid and lactic acid;
dyes for coloring the agent;
active ingredients such as bisabolol;
ceramides. Ceramides shall be understood to mean N-acylsphingosine (fatty acid amides of sphingosine) or synthetic analogs of such lipids (known as pseudoceramides);
blowing agents such as propane-butane mixtures, N₂O, dimethylether, CO₂, and air;
antioxidants;
pearlizing agents such as EGDS or PEG-3 Distearate;
preservatives, such as sodium benzoate or salicylic acid;
viscosity regulators such as salts (NaCl).

A second subject matter of the present invention is the cosmetic use of the hair cleansing agent according to the invention for improving detanglability and combability, in particular of damaged hair, and for reducing the static charge (“flyaway”) of hair and the development of shaggy or undesirably frizzy hair (“anti-frizz effect”).

What was said with respect to the agent according to the invention applies, mutatis mutandis, with respect to preferred embodiments of the use according to the invention.

### Examples

1) Production of Shampoos According to the Invention (Amounts in [% by Weight]):

| | Ex. 1 | Ex. 2 | Ex. 3 | Ex. 4 | Ex. 5 |
|---|---|---|---|---|---|
| Sodium Laureth Sulfate | 9.0 | 9.5 | 8 | 9 | 10 |
| (AS) | | | | | |
| Disodium | 0.8 | | 1 | 0.5 | 0.5 |
| Cocoamphodiacetate | | | | | |
| (AS) | | | | | |
| Cocoamidopropyle | 1.6 | 2.5 | 2 | 1.5 | 1.5 |
| Betaine (AS) | | | | | |
| Cocamide MEA | 0.9 | 0.5 | 0.6 | 0.5 | 0.7 |
| PEG-7 Glyceryl Cocoate | 0.4 | 0.6 | 0.5 | | 0.7 |
| PEG-40 Hydrogenated | 0.3 | 0.3 | 0.3 | 0.4 | 0.4 |
| Castor Oil | | | | | |
| Polyquaternium-10 | 0.5 | 0.2 | 0.4 | 0.1 | 0.3 |
| Guar | | 0.3 | | 0.2 | |
| Hydroxypropyltrimonium | | | | | |
| Chloride | | | | | |
| PEG-12 Dimethicone | 1.0 | 0.8 | | 0.7 | 1.0 |
| Panthenol | 0.2 | 0.2 | 0.1 | 0.2 | |
| Niacinamide | | | 0.1 | | 0.1 |
| Marula oil | | 0.05 | | | 0.05 |
| Jojoba oil | | | 0.1 | | |
| Apricot kernel oil | 0.1 | | | 0.1 | |
| Benzophenone-4 | 0.1 | 0.1 | 0.1 | 0.1 | 0.1 |
| Hydrogenated castor oil | 0.1 | 0.15 | 0.2 | 0.1 | 0.1 |
| Cocodimonium | 0.05 | 0.05 | 0.05 | | 0.05 |
| Hydroxypropyl | | | | | |
| Hydrolyzed Keratin | | | | | |
| Sodium benzoate | 0.5 | 0.5 | 0.5 | 0.5 | 0.5 |
| Lactic acid 80% DAB | 2.4 | 2.5 | 2.2 | 2.6 | 2.4 |
| Calcium hydroxide | 0.55 | 0.6 | 0.5 | 0.65 | 0.55 |
| Perfume | 0.5-1 | 0.5-1 | 0.5-1 | 0.5-1 | 0.5-1 |
| Water | to make | to make | to make | to make | to make |
| | 100 | 100 | 100 | 100 | 100 |
| pH value | 4.5-4.75 | 4.5-4.75 | 4.5-4.75 | 4.5-4.75 | 4.5-4.75 |
| | (±0.02) | (±0.02) | (±0.02) | (±0.02) | (±0.02) |

2) Evaluation of Shampoos According to the Invention

In a side-by-side experiment, one half of damaged, long hair was washed in the customary manner with a shampoo according to the invention (FDH14CB0003/13), which corresponds to above Example 1. The other half was washed in the customary manner with the same amount of a conventional nourishing shampoo (FDH14CB0003/12). The composition of the conventional nourishing shampoo likewise corresponded to above Example 1, except that citric acid (instead of lactic acid) was used to acidify the shampoo and the shampoo did not include calcium salt. Both shampoos had a pH value of 4.75. After the shampoos were rinsed off with water, the properties of the wet and dry hair listed below were compared to each other and rated simultaneously by 10 experts on a scale from 0 to 2.5 (differences>0.5 are significant; the formulation showing the reading in the diagram on its side was rated better by the experts in terms of the respective property).

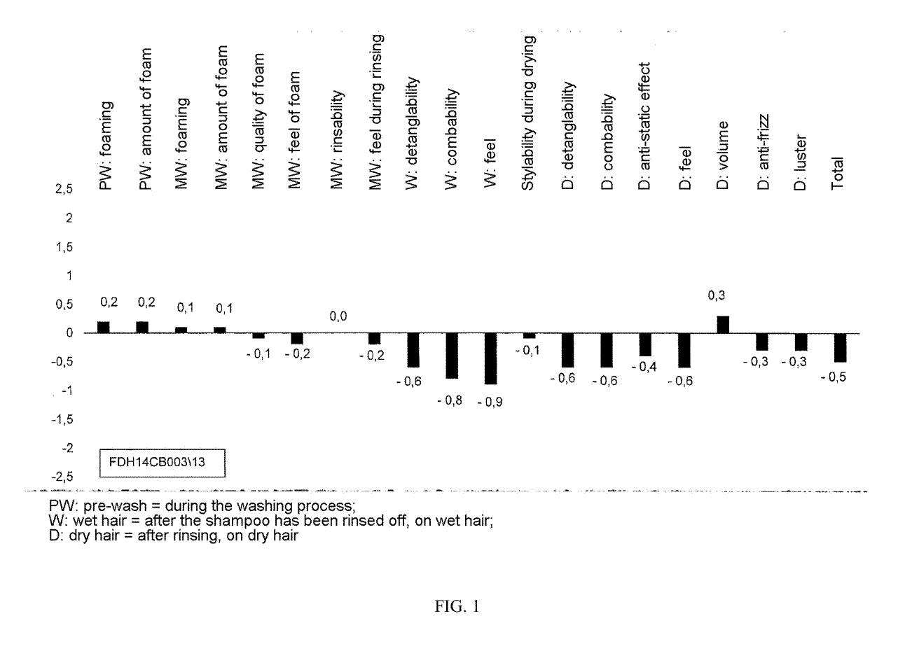

This is illustrated by FIG. 1.

It is clearly apparent from the diagram that half the hair, which was washed with the shampoo according to the invention, has noticeably better nourishing properties when wet and when dry.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A hair cleansing agent, comprising, based on the weight thereof:
a) 6 to 12 wt. % of at least one anionic surfactant;
b) 0.1 to 5 wt. % of at least one non-ionic surfactant;
c) 0.01 to 2 wt. % of at least one cationic nourishing polymer; and
d) (i) 0.01 to 3 wt. % calcium lactate or
(ii) 0.1 to 5 wt. % lactic acid and 0.05 to 3 wt. % of at least one inorganic calcium salt,
wherein the hair cleansing agent has a pH value in the range of 4.3 to 5.0.

2. The hair cleansing agent according to claim 1, wherein the agent has a pH value in the range of 4.4 to 5.0.

3. The hair cleansing agent according to claim 1, wherein the anionic surfactant is selected from the group consisting of ether carboxylic acids of formula R—O—(CH₂—CH₂O)ₓ—CH₂—COOH, in which R denotes a linear or branched, saturated or unsaturated alkyl group having 8 to 30 carbon atoms, and x=0 or 1 to 16, acyl isethionates having 8 to 24 carbon atoms in the acyl group, sulfosuccinic acid monoalkyl and/or dialkyl esters having 8 to 24 carbon atoms in the alkyl group, and sulfosuccinic acid monoalkyl polyoxyethyl esters having 8 to 24 carbon atoms in the alkyl group and 1 to 6 oxyethyl groups and from alkyl sulfate salts and/or alkyl polyglycol ether sulfate salts of formula R—(OCH₂—CH₂)ₓ—OSO₃₋X⁺, in which R denotes a linear or branched, saturated or unsaturated alkyl group having 8 to 30 carbon atoms, x denotes the number 0 or 1 to 12, and X⁺ denotes an alkali, alkaline earth, ammonium or alkanolamine ion.

4. The hair cleansing agent according claim 1, wherein the agent includes, based on the total weight thereof, 6 to 12 wt. % of at least one anionic alkyl ether sulfate salt of formula R—(OCH₂—CH₂)ₓ—OSO₃₋X⁺, in which R denotes a straight-chain or branched alkyl group having 8 to 18 carbon atoms, x denotes 1 to 6 ethylene oxide units, and X⁺ denotes a sodium, magnesium and/or triethanolamine ion.

5. The hair cleansing agent according to claim 1, wherein the non-ionic surfactant is selected from the group consisting of C₈ to C₃₀ fatty acid monoesters and/or diesters of addition products of 1 to 30 moles ethylene oxide to glycerol, addition products of 2 to 50 moles ethylene oxide and/or 0 to 5 moles propylene oxide to linear and branched fatty alcohols having 8 to 30 carbon atoms, fatty acid alkanolamides of the general formula 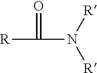
in which R preferably denotes a linear or branched, saturated or unsaturated alkyl or alkenyl group having 8 to 24 carbon atoms, and the groups R′ denote hydrogen or the group —(CH₂)ₙOH, in which n denotes the number 2 or 3, with the proviso that at least one of the groups R′ denotes the aforementioned group —(CH₂)ₙOH, and/or alkyl polyglucosides.

6. The hair cleansing agent according to claim 5, wherein the agent includes, based on the weight thereof, 0.1 to 5 wt. % of at least one fatty acid alkanolamide of the general formula

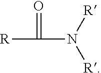

7. The hair cleansing agent according to claim 1, wherein the cationic nourishing polymer is selected from the group consisting of quaternized cellulose polymers, hydrophobically modified quaternized cellulose polymers, cationic guar derivatives, polymeric dimethyll diallyl ammonium salts and the copolymers thereof with esters and/or amides of acrylic acid and methacrylic acid, copolymers of vinylpyrrolidone with quaternized derivatives of dialkylaminoalkyl acrylate and methacrylate and/or polyvinylpyrrolidone.

8. The hair cleansing agent according to claim 1, wherein the agent includes, based on the weight thereof, 0.01 to 2 wt. % of at least one quaternized cellulose polymer.

9. The hair cleansing agent according to claim 8, wherein the at least one quaternized cellulose polymer is a cationic polymer obtainable by reacting hydroxyethyl cellulose with trimethylammonium-substituted epoxides and known under the INCI name Polyquaternium-10.

10. The hair cleansing agent according to claim 1, including calcium chloride, calcium hydroxide, calcium sulfate and/or calcium oxide as the inorganic calcium salt d) (ii).

11. The hair cleansing agent according to claim 1, wherein the agent includes, based on the total weight thereof,
i. 0.01 to 3 wt. % calcium lactate or
ii. 0.1 to 5 wt. % calcium hydroxide.

12. The hair cleansing agent according to claim 1, wherein the agent includes, based on the total weight thereof,
a) 6 to 12 wt. % of at least one anionic alkyl ether sulfate salt of formula R—(OCH₂—CH₂)ₓ—OSO₃₋X⁺, in which R denotes a straight-chain or branched alkyl group having 8 to 18 carbon atoms, x denotes 1 to 6 ethylene oxide units, and X⁺ denotes a sodium, magnesium and/or triethanolamine cation,
b) 0.1 to 5 wt. %/o of at least one fatty acid alkanolamide of formula 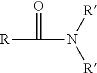
in which R denotes a linear or branched, saturated or unsaturated alkyl or alkenyl group having 8 to 24 carbon atoms, and the groups R′ denote hydrogen or the group —(CH₂)ₙOH, in which n denotes the number 2 or 3, with the proviso that at least one of the groups R′ denotes the aforementioned group —(CH₂)ₙOH;
c) 0.01 to 2 wt. % of at least one quaternized cellulose polymer, and
d) (i) 0.01 to 3 wt. % calcium lactate or
(ii) 0.1 to 5 wt. % lactic acid and 0.05 to 3 wt. % calcium hydroxide,
wherein the hair cleansing agent has a pH value in the range of 4.3 to 5.0.

13. The hair cleansing agent according to claim 1, further including, based on the total weight thereof, 0.01 to 5 wt. % of at least one silicone and/or at least one vegetable oil.

14. The hair cleansing agent according to claim 1, further including, based on the total weight thereof, 0.001 to 2 wt. % of at least one vitamin, vitamin derivative and/or vitamin salt and/or 0.001 to 5 wt. % of at least one protein hydrolysate.

15. The hair cleansing agent according to claim 1, further including, based on the total weight thereof 0.01 to 0.5 wt. % of at least one water soluble UV filter.
